(19) United States
(12) Patent Application Publication
(10) Pub. No.: US 2018/0214236 A1
(43) Pub. Date: Aug. 2, 2018

(54) Medical Consultation Stickers and Method of Use

(71) Applicant: Dawn Marie Leiva, (US)

(72) Inventor: Dawn Leiva, Rancho Santa Margarita, CA (US)

(21) Appl. No.: 15/880,461

(22) Filed: Jan. 25, 2018

Related U.S. Application Data
(60) Provisional application No. 62/450,547, filed on Jan. 25, 2017.

(51) Int. Cl. A61B 90/00; G06Q 90/00

(52) U.S. Cl. CPC A61B 90/08; A61B 2090/0807; G06Q 90/00

(57) ABSTRACT

Stickers for use in medical consultation and the method of use of such stickers is disclosed. While providing medical consultation for cosmetic procedures such as reducing fat via laser, radiation, or other energy emitting technology, doctors must demonstrate to their patients the region of the patient's body suitable for treatment. The stickers provide an avenue by which doctors and their patients can visualize the various potential treatment areas during consultation. Moreover, the stickers prevent the need for doctors to utilize a pen or marker to write on patients for visualization of the treatment area and prevent the need for patients to wash ink off their bodies. The stickers also preserve patient privacy by easily peeling off. The stickers comprise an outer perimeter 11, 21, 31, 41, 51, 61, 71 and at least one inner perimeter 12, 22, 32, 42, 52, 62, 72 with a surface area within the inner perimeter consisting of empty space 10, 20, 30, 40, 50, 60, 70 which displays the treatment area. On one side of the sticker there may be an adhesive to attach the sticker to a patient for medical consultation. Additionally, the stickers may contain holes between the inner and outer perimeter which serve as attachment points 14, 24, 34, 44, 54, 64, 74 by which the doctor can position the relevant medical treatment device.

### CROSS REFERENCE TO RELATED APPLICATIONS

This application claims priority to U.S. provisional application No. 62/450,547 filed Jan. 25, 2017, the contents of which are hereby incorporated by reference.

### BACKGROUND

During a medical consultation for cosmetic procedures the doctor often visualizes the patient's treatment area by writing on the patient with a pen or marker. This results in unwanted and difficult-to-clean ink on the patient. Moreover, the use of such ink compromises the privacy of the patient as others in the public or the patient's private life may view the consultation marks. Additionally, doctors have lacked a method by which to accurately measure the cost of treating multiple body areas during medical consultation. Because medical cosmetic work often contemplates cost based on the surface area of the body requiring treatment, the lack of a convenient measuring and visualization tool has impaired the ability of doctors to adequately and efficiently charge patients for their medical services. The same inability to visualize the discrete units of area that require treatment and to assess the concrete, associated costs has led patients to avoid cosmetic treatment. For example, a patient might refuse treatment because the costs of treating large, generalized parts of the body are too high. In contrast, the medical consultation stickers disclosed herein allow patients and doctors to collaboratively place stickers only where treatment is desired and accurately assess only those costs.

The relevant art discloses several methods by which doctors may visualize patient treatment areas. However, most of the relevant art discloses a method by which doctors can visualize the treatment area with the use of some form of ink or warning label. While prior stickers or adhesive labels have been designed and used for visualization and demarcation during surgery, none are designed for use during consultation prior to surgery.

Indeed, U.S. patent application Ser. No. 10/452,337 by Khalaj discloses a set of surgical marking strips overlay and the method of use of the overlay. The marking strips are pre-printed with desired surgical markings on one side preferably dashed lines, and on the other side can be bonded with adhesive that can be placed on the patient's skin where applicable for surgical procedure.

However, patent application Ser. No. 10/452,337 never discloses overlays, labels, or stickers which do not contain printed material. Instead, Khalaj discloses thin strips of stickers with printed ink meant to convey to a doctor during surgery where treatment is required. Khalaj does not disclose stickers for use during a medical consultation having an open surface area in the middle of the overlay which measures the space requiring later medical treatment and thus assesses the cost of said treatment.

Moreover, U.S. Pat. No. 4,869,531 by Rees discloses an apparatus and method for documenting the findings of a physical examination. A group of pre-printed stickers are provided having an anatomical designation (either graphic or text) on the front surface of the sticker and an adhesive and peel-off back sheet on the rear of the sticker. The stickers are positioned in a dispenser with at least a portion of the anatomical designation visible from the sticker to facilitate selection of the desired stickers during the examination. The physician or patient can mark directly on the sticker to indicate the location, size and shape of any abnormality, and the back sheet is then removed and the sticker attached directly onto the patient's progress report. The system is an inexpensive method for fully documenting all findings from an examination in a minimum period of time and provides a permanent record which facilitates retrieval of the desired information from the report and minimizes the volume of patient records.

However, U.S. Pat. No. 4,869,531 is completely distinguishable from the claimed invention. U.S. Pat. No. 4,869,531 merely provides for stickers with printed materials conveying some association with the human anatomy which are attached to a patient's medical file to streamline the medical recording process. U.S. Pat. No. 4,869,531 does not disclose stickers which are adhered to patients in order to visualize treatment areas and assess the associated costs.

Furthermore, U.S. Pat. No. 6,524,250 by Weber discloses a device that can be easily used by surgeons to measure and monitor changes before, during, and after a liposuction procedure and assist in producing symmetrical body contours. The device comprises a remote control and data processing unit, a handheld ultrasound transducer, a display monitor and means for marking anatomical points to be measured. However, this invention does not include the use of stickers to visualize treatment surface area but rather the use of an electronic device to monitor tissue thickness specifically.

Additionally, U.S. Pat. No. 7,127,826 B2 by Russell discloses markers, methods of marking, and marking systems for use in association with images are disclosed. One marker is removable with a targeted design and metric dimensions inscribed on a clear plastic flap. This marker is attached adjacent to an area of interest with the target placed in such a manner to precisely pin point out an observed abnormality. A paper label at one edge is available for written comments and directions for further studies. The marker can be removed without damage to the image(s). But, U.S. Pat. No. 7,127,826 does not involve stickers with empty surface area for the visualization of treatment areas. Instead, U.S. Pat. No. 7,127,826 involves various markers which merely designate possible abnormalities via a pinpoint mark. Also, the method in U.S. Pat. No. 7,127,826 regards a method in which medical imagery is marked, whereas the claimed method herein regards placing stickers on patient bodies.

Next, U.S. patent application Ser. No. 11/221,180 by Moosheimer et al., discloses a strip-shaped label has a transparent plastic film layer partially covered by an opaque film layer. A window area not covered by the opaque film layer remains. The film layers are bonded to one another using an adhesive layer. Before attachment, the label is positioned on a carrier. A bow-shaped suspension tab is produced through punching, on whose bottom non-adhesive areas are provided, which allow a suspension tab to be pulled out of the label plane easily for suspension of the container. Two printed voucher sections are implemented in the opaque label film. The transparent film layer has an adhesive-repellent coating, which is also transparent, below the voucher sections. In the stuck-on state, the label area in which the voucher sections are positioned overlap the transparent window area. After removal of one of the voucher sections, the window area is visible and the container contents may be viewed. However, this application regards labels most suitable for pharmaceutical containers and thus does not provide a large open surface area for visualization of cosmetic treatment on patient bodies.

Further, U.S. Pat. No. 8,016,323 B2 by Hunt discloses a self-adhesive label that is provided on a web of siliconized polyester liner and that has a base label with an upper printable surface and a lower adhesive coated surface and at least one patch of release material located between the base label and the web of siliconized polyester liner. By forming the patches of release material on the adhesive surface of the base label and then applying a siliconized polyester liner over the patches and the exposed adhesive surface of the base label, the resultant self-adhesive label is more robust and as the siliconized polyester liner has a substantially continuous upper surface, the risks of snagging as a result of edges and windows in the web of backing material is avoided. However, U.S. Pat. No. 8,016,323 B2 merely discloses a type of self-adhesive label without an open surface area suitable for cosmetic treatment visualization.

Similarly, U.S. Pat. No. 8,608,204 B2 by Brennan et al., discloses a self-attaching tag made essentially of self-adhesive printable plastic to be attached to a fragile and easily deformable object characterized in that the tag is covered with a plurality of varnishes which allow for the protection of pre-printing and for printing and writing on at the moment of use. The tags are delivered on large rolls where perforations allow for easy separation of the tags. However, again, the tags are completely opaque and most suitable with regard to their size for fluid bags.

Additionally, U.S. patent application Ser. No. 10/143,758 by Adolph et al., discloses a method of capturing medical information. Capturing medical information concerning patients is an important task for medical practitioners because the information generally allows the practitioners to track their patient's medical history. However, depending on individuals to manually record medical information such as replacement part codes, may result in human error. In order to reduce the chance of human error, a PDA scanner is used to scan and store the medical information into a PDA. The scanned information may then be immediately entered into a medical information record corresponding to the patient undergoing surgery. However, application Ser. No. 10/143,758 regards electronic storage of patient information and does not pertain to stickers used for assessing cosmetic treatment costs on patient bodies.

Moreover, U.S. patent application Ser. No. 10/423,803 by Bruce discloses a surgical safety device and method, utilizing one or more indicators or markers which show a surgeon where to operate, in which the markers are specifically intended to eliminate errors which arise when a surgeon fails to select the correct side for conducting a surgical procedure on his or her bilaterally symmetrical patient, in which such markers positively and unambiguously identify the correct side of the patient upon which to operate, especially when combined in a procedure or protocol requiring the use of two markers. However, application Ser. No. 10/423,803 discloses labels which convey to the doctor via printed information which side of a patient's body requires surgery. It does not disclose a sticker used in a medical consultation with an open surface area for visualizing and assessing the cost of a cosmetic treatment site.

Similarly, U.S. patent application Ser. No. 10/444,632 by Hered discloses a method of stamping a patient's body to convey medical information and a kit for accomplishing the method. The method provides for positioning medical information related to a medical procedure to be performed at a location on the patient's body related to the procedure to be performed. Again, it does not disclose a sticker with an open surface area for visualizing and assessing the cost of a cosmetic treatment site.

U.S. patent application Ser. No. 11/109,080 by Seidl discloses a label having a base layer, on which at least one product information sheet is positioned, and having an upper label layer, which is positioned on the base layer and is shaped in such a way that it forms at least one hanger loop and a cover sheet covering the at least one product information sheet. In the novel label, according to the present invention, the cover sheet and the hanger loop are manufactured from one single material layer, specifically the upper label layer. The label is therefore distinguished in relation to the typical achievements of the object by efficient and material-saving manufacturing. As a further advantage, it is to be noted that the individual components, such as hanger loop and cover sheet, do not mutually influence one another during use. However, again, application Ser. No. 11/109,080 does not disclose stickers for visualizing cosmetic treatment area but rather an opaque label designed for conveying product information on medical infusion bags.

Next, U.S. patent application Ser. No. 13/160,546 by Dehlinger et al., discloses an invention relating to a pattern coated pressure sensitive label assembly for use in preserving patient privacy in medical treatment bag applications. The label assembly utilizes specific zones and areas to create removable sections that will permit areas containing patient information to be separated from the remainder of the label which remains with the treatment bag during disposal. Therefore, U.S. patent application Ser. No. 13/160,546 does not address visualizing a medical treatment area.

Moreover, U.S. Pat. No. RE42,531 E by Brown et at., discloses a container made from an elongated sheet having a front surface, a rear surface, and a first end, a second end, a top edge and a bottom edge. The sheet includes a removable portion and a remainder portion. Both the removable portion and the remainder portion have identifying indicia printed thereon. The elongated sheet is preferably made of an uniaxially oriented material. The rear surface has an adhesive coating applied thereto. An adhesive reducing coating is applied to the adhesive coating adjacent to the first end of the elongated sheet. However, U.S. Pat. No. RE42,531 E regards the use of detachable stickers on pharmaceutical products wherein the detachable portions can be re-adhered in a medical record for efficient record keeping. It, and the other relevant art cited herein, do not address the problem of visualizing cosmetic treatment area and assessing the related costs for patients.

### SUMMARY OF THE INVENTION

It is an object of the invention to efficiently and accurately visualize the treatment area of a patient seeking cosmetic surgeries during a medical consultation.

It is another object of the invention to more efficiently assess the cost of a patient's cosmetic treatment by associating a particular treatment cost with the application of each sticker on the patient's body during a medical consultation.

It is yet another object of the invention to ensure that cosmetic medical consultations remain private between the physician and patient by avoiding the use of pens and markers on the patient's body.

The invention comprises a sticker having an outer perimeter and at least one inner perimeter, the surface area within the inner and outer perimeters forming a sticker band. Within the inner perimeter is an open surface area which visualizes the treatment area on the patient's body once affixed thereto. In one embodiment, the sticker is manufactured affixed to a piece of paper and the physician removes the sticker from said paper in order to attach the sticker to the patient via an adhesive on one side of the sticker. The sticker may contain attachment points for adjusting the position of the treatment device upon the patient's body.

In another embodiment, there is a repositionable pressure-sensitive adhesive on one side of the sticker such that the sticker may be taken off the patient's body and attached elsewhere on the patient's body.

In another embodiment, the sticker contains a very light adhesive such that the sticker cannot be reused after it is attached once to a patient, preserving sanitation.

In yet another embodiment of the invention, the sticker is manufactured with perforations such that the sticker tears after being attached once to a patient, preserving sanitation.

### BRIEF DESCRIPTION OF THE DRAWINGS

FIG. 1 is a plan view showing a preferred embodiment of a sticker having two treatment area spaces wherein the long axis of the treatment area space is horizontally positioned.

FIG. 2 is a plan view showing a preferred embodiment of a sticker having two treatment area spaces wherein the long axis of the treatment area space is vertically positioned and there is a large band surface area separating the treatment area spaces.

FIG. 3 is a plan view showing a preferred embodiment of a sticker having two treatment area spaces wherein the long axis of the treatment area space is vertically positioned and there is a smaller band surface area separating the treatment area spaces.

FIG. 4 is a plan view showing a preferred embodiment of a sticker having four treatment area spaces wherein the long axis of the treatment area space is horizontally positioned.

FIG. 5 is a plan view showing a preferred embodiment of a sticker having one treatment area space wherein the long axis of the treatment area space is vertically positioned.

FIG. 6 is a plan view showing a preferred embodiment of a sticker having one treatment area space wherein the long axis of the treatment area space is horizontally positioned.

FIG. 7 is a plan view showing a preferred embodiment of a sticker having three treatment area spaces wherein the long axis of the treatment area space is horizontally positioned.

### DETAILED DESCRIPTION OF THE INVENTION

The invention disclosed herein, medical consultations stickers and the method of their use, allows for accurate visualization of a cosmetic treatment area during medical consultations. Moreover, the stickers allow for accurate and efficient cost assessment of treatments. Whereas in the past physicians associated treatment costs with generalized regions of the human body, with this invention physicians can precisely associate particular costs with the treatment area space 10, 20, 30, 40, 50, 60, 70. Additionally, the patient can more selectively choose her treatment with the clear visualization of the treatment area space 10, 20, 30, 40, 50, 60, 70, rather than accept treatment on large, generalized areas of the body. Moreover, because physicians typically use a pen or marker to visualize cosmetic treatment, it is often apparent to the public or patient's spouse that the patient underwent a medical consultation as such pen or marker marks are difficult to clean off. By using the stickers disclosed herein, the patient can preserve his or her privacy by avoiding the need to be marked by a pen or marker during consultation.

The medical consultation sticker comprises at least one inner perimeter 12, 22, 32, 42, 52, 62, 72 and an outer perimeter 11, 21, 31, 41, 51, 61, 71, with open surface area within the inner perimeters--the treatment area space 10, 20, 30, 40, 50, 60, 70. The surface area between the inner and outer perimeters is the sticker band 13, 23, 33, 43, 53, 63, 73, which is attached to the patient during consultation.

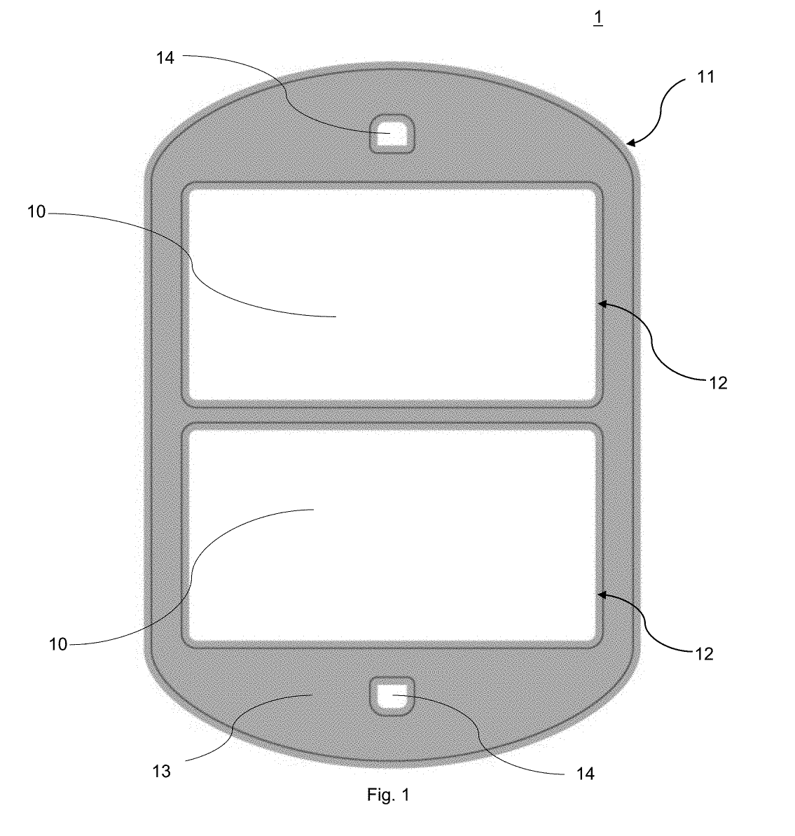

FIG. 1 depicts one preferred embodiment of the invention, wherein the medical consultation sticker has two rectangular treatment area spaces 10. In this embodiment, the treatment area spaces 10 have their long axes positioned horizontally. Moreover, the sticker comprises inner perimeters 12 and an outer perimeter 11, with the surface area between the inner and outer perimeters forming the sticker band 13 which is attached to the patient during medical consultation. Additionally, FIG. 1 contains two attachment points 14 which may be used by the physician for positioning and aligning the relevant medical device.

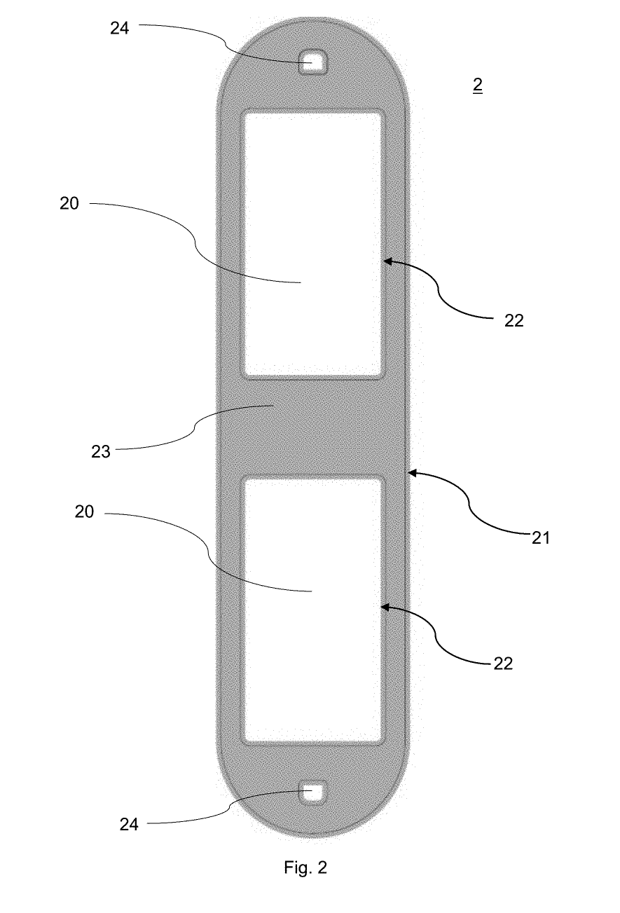

FIG. 2 depicts another preferred embodiment in which the invention comprises two treatment area spaces 20. In this embodiment, the long axis of the treatment area space is positioned vertically and there is a large surface area gap between the two treatment area spaces 20. This embodiment is particularly ideal for consultation regarding the navel area.

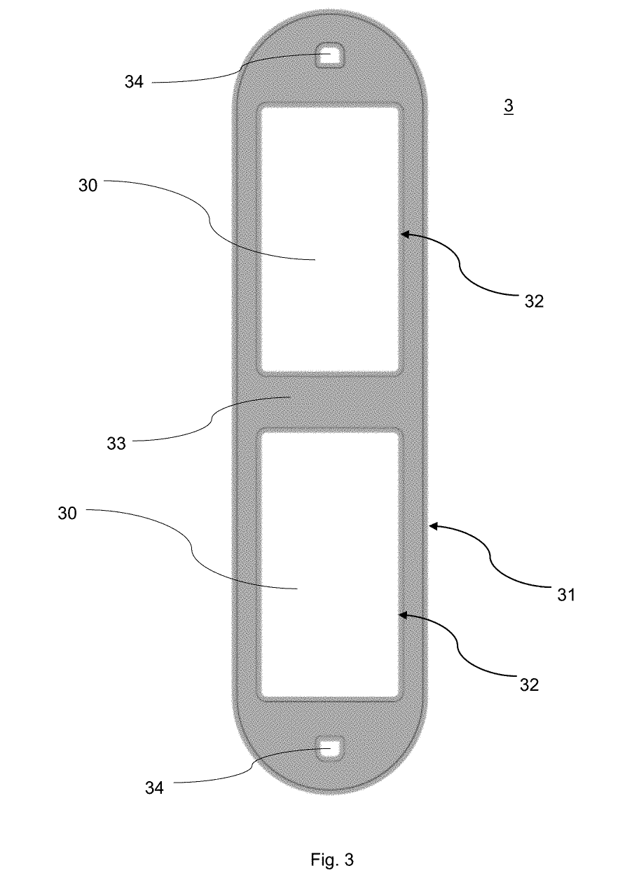

FIG. 3 is similar to FIG. 2, except that the surface area between the two treatment area spaces 30 is relatively smaller than the same surface area in FIG. 2.

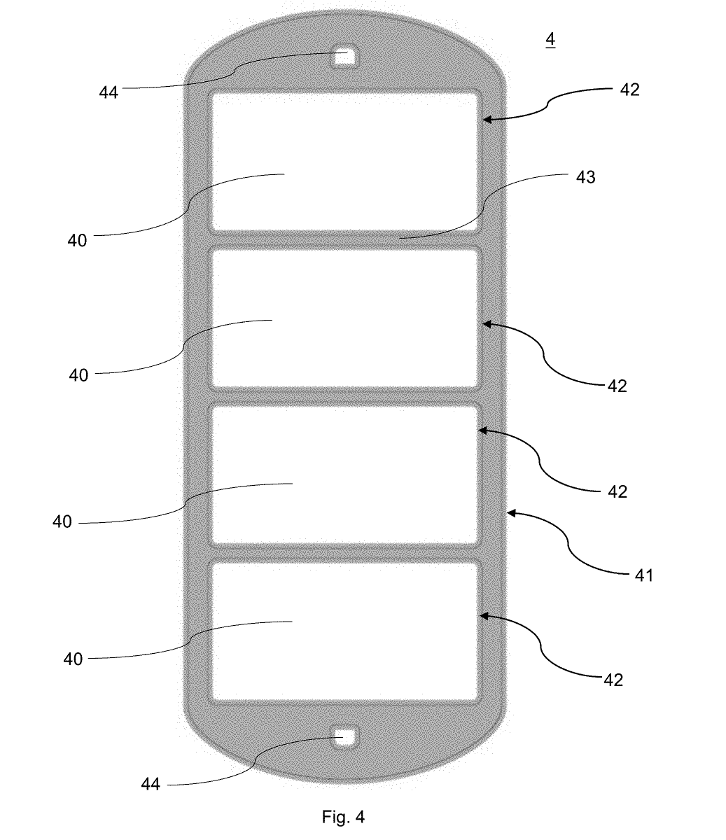

FIG. 4 depicts an embodiment in which there are four treatment area spaces 40 which have their long axes positioned horizontally.

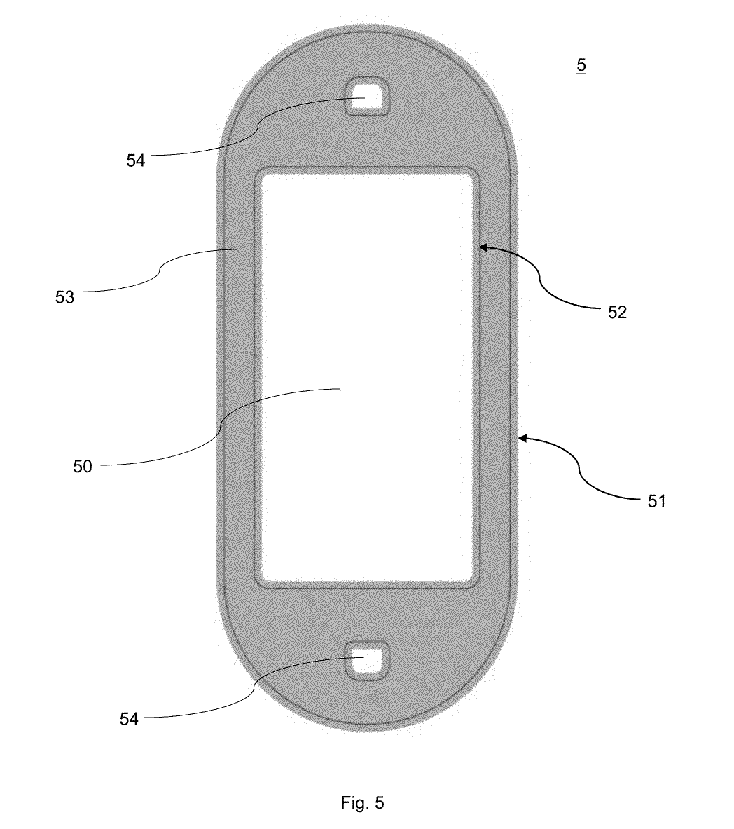

FIG. 5 depicts an embodiment in which there is one rectangular treatment area space 50 with a vertically positioned long axis.

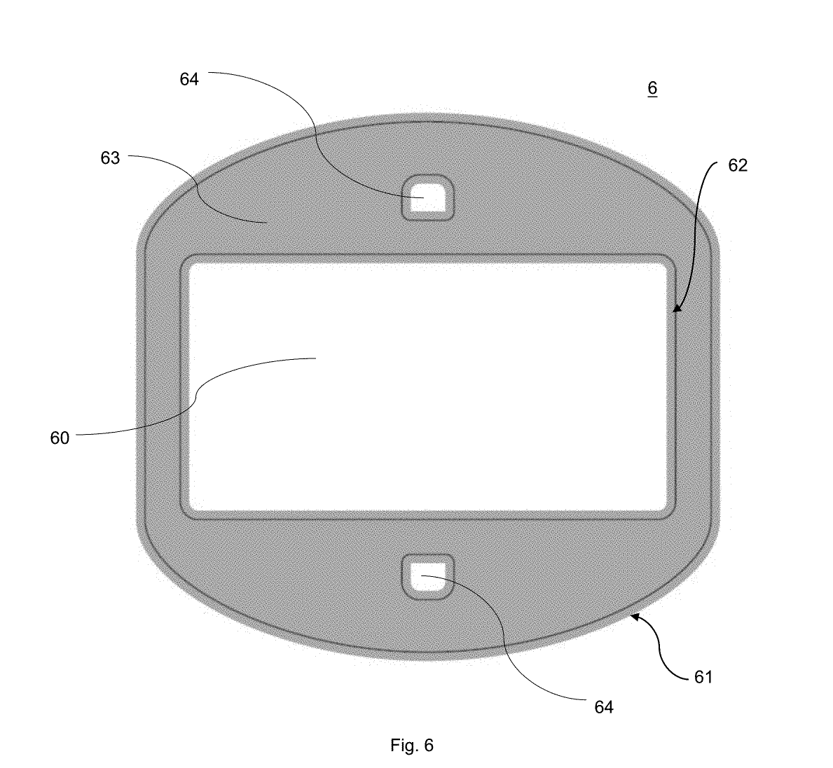

FIG. 6. Depicts an embodiment in which there is one rectangular treatment area space 60 with a horizontally positioned long axis.

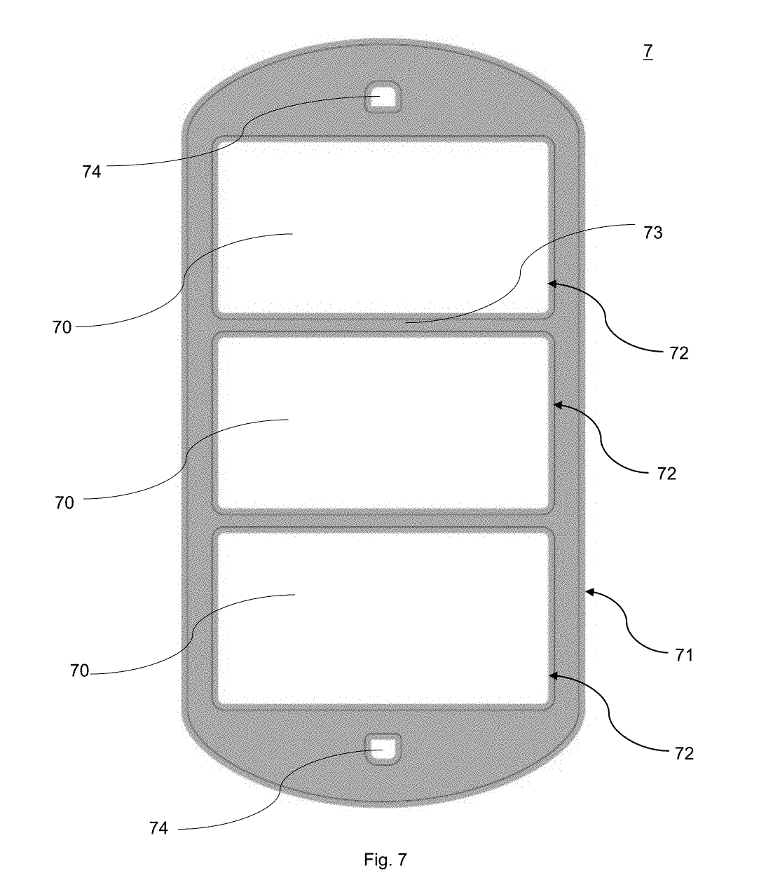

Lastly, FIG. 7 depicts an embodiment in which there are four rectangular treatment area spaces 70 with horizontally positioned long axes.

In many embodiments, the invention may have attachment points 14, 24, 34, 44, 54, 64, 74 used by the physician in positioning and aligning the relevant medical device used in the cosmetic treatment. The medical consultation stickers are ideal for use with devices used in coolscultping®, Zerona®, truSculpt®, Vanquish®, and devices used in other fat contouring procedures.

In order to affix the sticker on the patient, the sticker can be manufactured with an adhesive on one side. The sticker having adhesive may come initially affixed to a piece of paper. Alternatively, the sticker may have no adhesive and instead be adhered to the patient via moisture.

The stickers can also be manufactured to ensure that they are not reused on subsequent patients in order to preserve cleanliness in the consultation process. For example, in one embodiment, the stickers contain a very light adhesive such that the sticker cannot be reused after the stickers are attached once to a patient. In yet another embodiment of the invention—which is not depicted in the figures herein—the sticker is manufactured with perforations in the sticker band such that the sticker tears after being attached once to a patient, and therefore, cannot be reused. In this embodiment, the perforations in the sticker band would comprise cuttings or thinning in the sticker band.

Alternatively, the sticker can be manufactured such that one sticker can be affixed multiple times on the same patient's body, but in different locations. This can be achieved by manufacturing the sticker with a repositionable biocompatible pressure-sensitive adhesive, for example a silicone gel based pressure-sensitive adhesive, on one side of the sticker that allows the sticker to be easily transferred from one part of a patient's body to another without compromising the sticker's adhesive property.

Moreover, the sticker band 13, 23, 33, 43, 53, 63, 73 can be manufactured out of many materials. For example, the sticker band can be made of paper, vinyl, cut vinyl, polypropylene, or polyester. The stickers can come individually, in sterilized kits with various designs, or on sterilized rolls.

With regard to the invention's ideal measurements and shape, many embodiments will suffice. For example, the sticker band's outer perimeter and/or inner perimeter(s) can be manufactured to be circular, triangular, rectangular, hexagonal, or any other desired two-dimensional shape. With regard to measurement, in one preferred embodiment the outer perimeter of the sticker band would be rectangular with a length ranging from 76 mm to 305 mm and a width ranging from 50 mm to 152 mm. Moreover, the treatment area space is preferably shaped as a rectangle with a length ranging from 25 mm to 102 mm and also a width ranging from 25 mm to 102 mm. Additionally, the sticker preferably has a band thickness of 1 mm to 3 mm. Lastly, should the sticker contain attachment points, the dimensions of such attachment points would preferably measure about 8.5 mm in both width and length; if the attachment point is circular, the diameter of the attachment point would preferably be about 8.5 mm.

Moreover, with regard to the method of use of the medical consultation stickers, first, the physician associates the treatment area space of each sticker with a particular treatment cost. The physician then discusses the potential treatment areas with the patient and affixes stickers only in those areas of interest. Stickers may be added or removed from the patient's body as the physician discuss potential treatment areas. Should the stickers be manufactured with a repositionable adhesive agent, the stickers can be taken off the patient's body and moved to a more appropriate location on the body as deemed by the physician or patient. The sticker bands are placed on the patient body in such a way that the treatment area spaces expose and visualize the areas of the patient's body for which treatment is desired. Once the entirety of the treatment site(s) is selected and visualized via the treatment area spaces of the stickers, the physician can assess the total cost of the treatment by summing the associated treatment cost of each sticker placed on the patient's body. For example, if the physician associates a treatment area space with a width and length of 100 mm with $500 worth of treatment costs, and the patient has two stickers each having one treatment area space with the aforementioned dimensions affixed to him or her, then the total treatment cost would be summed to $1000. Likewise, if the physician associates a treatment area space having a width of 50 mm and length of 75 mm with a treatment cost of $250, and the patient is wearing one sticker with four such treatment area spaces, then the total cost of the treatment would be summed to $1000. The stickers can be removed from the patient's body by either the physician or patient after the medical consultation by peeling the stickers off of the body. Alternatively, should the stickers contain attachment points, the physician can initially align the relevant medical treatment device with the attachment point for cosmetic treatment.

The invention claimed is:

1. A medical consultation sticker comprising an outer perimeter and at least one inner perimeter, wherein the surface area within the inner perimeter, the treatment area space, is empty and serves to visualize a medical treatment area.

2. The medical consultation sticker of claim 1, wherein one side of the medical consultation sticker further comprises an adhesive agent.

3. The medical consultation sticker of claim 2, wherein the adhesive agent is a repositionable pressure-sensitive adhesive which allows said sticker to be reused without compromising its adhesive properties.

4. The medical consultation sticker of claim 1, 2, or 3, wherein the medical consultation sticker further comprises attachment points for aligning the relevant treatment device over the treatment area.

5. The medical consultation sticker of claim 1 or 2, wherein said medical consultation sticker is manufactured with perforations which tear after a single use of said medical consultation sticker.

6. A method of use of medical consultation stickers comprising the steps of: the physician associating a treatment cost with the treatment area space of each medical consultation sticker potentially used in the consultation; the physician discussing with the patient the areas of the body that the patient seeks treatment upon; the physician adhering the medical consultation stickers in such a way that the treatment area spaces expose and visualize the areas of the patient's body for which treatment is desired; removing and/or adding medical consultation stickers to the patient's body until only those areas that will be treated upon are visualized by the medical consultation stickers; summing the associated treatment cost of each sticker placed on the patient's body.

7. The method of claim 6, wherein, while discussing potential treatment areas, the medical consultation stickers already on the patient's body are removed and re-adhered on other regions of the body via a repositionable pressure-sensitive adhesive agent present on one side of said sticker, as decided by the patient and/or physician.

8. The method of claim 6 or 7, wherein the medical consultation stickers are removed by either the physician or patient after the medical consultation.

9. The method of claim 6 or 7, wherein, after consultation, the physician aligns the relevant treatment device over a medical consultation sticker having at least one attachment point.
